(19) United States
(12) Patent Application Publication
(10) Pub. No.: US 2003/0187069 A1
(43) Pub. Date: Oct. 2, 2003

(54) Treatment of burns

(76) Inventors: Dominique Sallin, Gland (CH); Jean-Luc Kienzler, Ornex (FR); Phyllis Schumann, Morges (CH); Jacek Ancerewicz, Morges (CH)

Correspondence Address: THOMAS HOXIE, NOVARTIS, CORPORATE INTELLECTUAL PROPERTY, ONE HEALTH PLAZA 430/2, EAST HANOVER, NJ, 07936-1080, US

(21) Appl. No.: 10/363,497

(22) PCT Filed: Aug. 30, 2001
(86) PCT No.: PCT/EP01/10041

(30) Foreign Application Priority Data
Sep. 1, 2000 (EP) 00118968.7

(51) Int. Cl. A61K 31/195

(52) U.S. Cl. 514/567

(57) ABSTRACT

The invention relates, to the topical use of diclofenac, and topically acceptable salt, thereof, (for the manufacture of a topical medicament) for the topical treatment of burns.

The invention relates to the topical (=external) treatment of burns including sunburn with diclofenac or a topically acceptable salt thereof.

The topical application of diclofenac, or topically acceptable salts thereof, for the treatment of e.g. back pain, muscle pain, sprains, bruises, lumbago, epicondylitis, osteoarthritis or rheumatic arthritis is known in the art.

It has now surprisingly been found that by topical application of diclofenac, or a topically acceptable salt thereof, burns of the skin including sunburns can be treated very effectively, which inter alia means that the healing process is promoted dramatically and that the distress of a patient suffering from a burn is alleviated rapidly.

Therefore, the invention relates to the use of diclofenac, or a topically acceptable salt thereof, (for the manufacture of a topical medicament) for the topical treatment of burns including sunburn.

Burns can be caused e.g. by radiation, e.g. sunburn, or e.g. by contact with hot solid objects, such as a hot plate, hot liquids, such as hot water, or hot gases.

Diclofenac is 2-(2,6-dichloroanilino)-phenylacetic acid (=diclofenac free acid). Topically applicable salts of diclofenac are e.g. diclofenac sodium, diclofenac potassium, diclofenac diethylammonium and diclofenac epolamine, with diclofenac diethylammonium, diclofenac epolamine and diclofenac sodium being preferred. Especially preferred are diclofenac diethylammonium and diclofenac sodium—in one particular embodiment diclofenac diethylammonium, and in another particular embodiment diclofenac sodium.

Diclofenac can be applied—typically in the form of a topical pharmaceutical composition—to any portion of the skin.

The beneficial properties of diclofenac when topically administered in the treatment of burns including sunburn can be demonstrated, for example, in the following tests.

(1) In 60 guinea pigs erythema of sunburn are induced by UV radiation [with different irradiation doses of 1, 5 and 10 MED (1 MED=minimal erythemal dose, i.e. the irradiation dose which is just sufficient to induce erythema)]. A topical formulation comprising 1.16% diclofenac diethylammonium [corresponding to 1% diclofenac sodium] (Voltaren® Emulgel®) is applied on the irradiated skin (either 2 mg/cm², 10 mg/cm² or 50 mg/cm²). The erythema is strongly reduced in a dose-related manner and significantly better than with placebo.

(2) In an analogous manner as described in (1), a topical test formulation comprising 1% diclofenac sodium is applied on the irradiated skin (either 2 mg/cm², 10 mg/cm² or 50 mg/cm²). The erythema is strongly reduced in a dose-related manner and significantly better than with placebo.

(3) In an analogous manner as described in (1), a topical test formulation comprising 0.29% diclofenac diethylammonium [corresponding to 0.25% diclofenac sodium] is applied on the irradiated skin (either 2 mg/cm², 10 mg/cm² or 50 mg/cm²). The erythema is strongly reduced in a dose-related manner and significantly better than with placebo.

(4) In an analogous manner as described in (1), a topical test formulation comprising 0.58% diclofenac diethylammonium [corresponding to 0.5% diclofenac sodium] is applied on the irradiated skin (either 2 mg/cm², 10 mg/cm² or 50 mg/cm²). The erythema is strongly reduced in a dose-related manner and significantly better than with placebo.

(5) Several cohorts of 25 hairless rats each are irradiated with UV radiation, and erythema of sunburn are induced in all rats. All rats are then treated with a topical formulation comprising 1.16% diclofenac diethylammonium (Voltaren® Emulgel®) but with the beginning of treatment being different in each cohort. It can be shown that the earlier treatment is started after UV radiation, the more quickly is the reversal of erythema.

(6) Hairless rats with erythema induced by UV radiation are treated with Voltaren® Emulgel® as described under (5). A control group of hairless rats with no erythema is likewise treated with Voltaren® Emulgel®. The total plasma concentration of diclofenac is determined in both groups. It can be shown that the concentration of diclofenac is essentially the same in both groups. So there is observed no increase of the systemic absorption of diclofenac, if diclofenac is applied to irradiated skin (as compared to non-irradiated skin).

The safety of the compositions of the invention is warranted inter alia by the long-time, proven use of topical diclofenac compositions in other indications, such as back and muscle pain, e.g. via the marketed product Voltaren® Emulgel® and many other topical formulations comprising either diclofenac sodium, diethylammonium or epolamine being on the markets.

In particular, the invention relates to the use of diclofenac, or a topically acceptable salt thereof, where the diclofenac component is present in an amount of from 0.01 up to 15%—preferably of from 0.1 up to 5%, especially of from 0.3 up to 3%, more especially of from 0.4 up to 2.5%, and first and foremost of from 0.5 up to 2%—of the total of the topical composition. A particular embodiment of the invention is characterized by the use of the diclofenac component—in particular diclofenac diethylammonium and diclofenac sodium, especially diclofenac sodium—in an amount of from 0.01 up to 2%, or of from 0.05 up to 1.3%, or of from 0.1 up to 2%, preferably of from 0.1 up to 1%, more preferably of from 0.1 up to 0.7% and most preferably of from 0.1 up to 0.5%, of the total composition. All percentages given are weight-% (w/w), if not indicated otherwise.

Preferably, said topical compositions comprise the diclofenac component in therapeutically effective amounts.

The dosage of the active ingredient may depend on various factors, such as sex, age and individual condition of the patient, as well as on the kind of burn involved. Typically, the topical pharmaceutical compositions—e.g. in the form of an emulsion-gel, gel, cream or ointment—are applied once, twice, three times or four times daily. What is important is that the treatment is started as early as possible after the burn has occurred. Typically, after a first application of topical diclofenac, one can wait for e.g. 3-4 hours before repeating the application. Transdermal patches and bandages comprising a diclofenac component also come into consideration as topical formulations. Those may be applied, for example, once per 16 hours, once daily or once per two or three days, with once per 16 hours or once daily being preferred.

Moreover, the invention relates to a method of treating burns including sunburn which comprises topically administering to a mammal in need of such treatment a therapeutically effective amount of diclofenac or a topically applicable salt thereof.

Pharmaceutical compositions suitable for topical administration are e.g. creams, lotions, ointments, microemulsions, fatty ointments, gels, emulsion-gels, pastes, foams, tinctures, solutions; transdermal therapeutic systems (TTS), in particular transdermal patches; plasters and bandages. Preferred are emulsion-gels, gels, creams, lotions, solutions, transdermal patches, plasters and bandages. In particular preferred are emulsion-gels, gels and transdermal patches, especially emulsion-gels and transdermal patches, and first and foremost emulsion-gels. Said compositions are all known in the art; for further details refererence is made e.g. to U.S. Pat. No. 4,551,475, columns 7-9 and U.S. Pat. No. 4,917,886, columns 10-12.

For example, emulsion-gels represent topical compositions which combine the properties of a gel with those of an oil-in-water emulsion. In contrast to gels, they contain a lipid phase which due to its fat-restoring properties enables the formulation to be massaged in whilst, at the same time, the direct absorption into the skin is experienced as a pleasant property. In contrast to gels which typically are clear and transparent, emulsion-gels are characterized by a turbid, opaque appearance.

For example, transdermal therapeutic systems (TTS's) contain the diclofenac component typically together with a carrier. Useful carriers may include absorbable, pharmacologically suitable solvents to assist passage of the active ingredient through the skin. The TTS's are, for example, in the form of a transdermal patch comprising (a) a substrate (=backing layer or film), (b) a matrix containing the diclofenac component, optionally carriers and optionally a special adhesive for attaching the system to the skin, and normally (c) a protection foil (=release liner). The matrix (b) is e.g. present as a mono-layer but may also consist of different layers.

The manufacture of the topical pharmaceutical preparations in general is known in the art. Likewise, examples of topical pharmaceutical compositions comprising diclofenac components are known in the art, see e.g. U.S. Pat. No. 4,917,886, example 1 (and examples 2-7 as well), or U.S. Pat. No. 4,551,475, examples 8-16, or EP 372 527 A1 (e.g. examples 1-6), or EP 621 263 A2 (e.g. examples 1-3).

### EXAMPLE 1

60 guinea pigs are irradiated by UV light (UV-B) with an irradiation dose of 10 MED (1 MED here corresponds to a radiant exposure of about 78 mJ/cm² during 1 min) to induce erythema. The irradiated area has a diameter of ca. 9 mm. After irradiation, the irradiated skin is treated with either Voltaren® Emulgel® (three different strengths: 2 mg, 10 mg or 50 mg diclofenac diethylammonium per cm²) or placebo. One hour after treatment the irradiated portions of the skin of the animals are inspected. The result is that all three dosages of Voltaren® Emulgel® are statistically significantly more potent than placebo (p<0.05) in reducing the erythema induced by 10 MED irradiation.

### EXAMPLE 2

A double-blind controlled clinical study is performed in 24 patients. After evaluation of individual MED's, each patient is irradiated by UV light (UV-B) to induce sunburn, with two different sites being irradiated in each case. The irradiated skin is treated with either Voltaren® Emulgel® or placebo. 1 and 2 hours after treatment, a statistically significant relief of UV induced pain (spontaneous and provoked pain) and erythema (visual score and chromatography) is observed in the patients treated with Voltaren® Emulgel®.

### EXAMPLE 3

A double-blind controlled clinical study is performed in 30 patients. After evaluation of individual MED's, each patient is irradiated by UV light (UV-B) to induce sunburn, with four different sites being irradiated in each case. The irradiated skin is treated with either a topical test formulation comprising 1% diclofenac sodium or placebo. What is measured is the time needed for recovery of the irradiated skin. Said time is statistically significantly shorter in the group treated with diclofenac sodium than in the placebo group. In contrast to the group treated with diclofenac sodium, at first a worsening of skin lesions including development of visible eodema and enlargement of erythema is observed in the placebo group.

1. Use of a pharmaceutically active substance selected from the group consisting of diclofenac sodium, diclofenac potassium, diclofenac dlethylammonium and diclofenac epolamine, for the manufacture of a topical medicament for the topical treatment of burns including sunburn,
which topical medicament is characterized in that the pharmaceutically active substance selected is not in conjunction with a solution of nitrous oxide in a pharmaceutically acceptable carrier solvent, and
which topical medicament is further characterized in that the pharmaceutically active substance selected is not part of a herbal formulation comprising plant parts or oils from medicinal plants.

2. Use according to claim 1, where diclofenac sodium is selected as pharmaceutically active substance.

3. Use according to either claim 1 or claim 2, where the pharmaceutically active substance is present in an amount of from 0.01 up to 2 weight-% of the total composition.

4. Use according to either claim 1 or claim 2, where the pharmaceutically active substance is present in an amount of from 0.01 up to 0.7 weight-% of the total composition.

5. Use according to any ones of claims 1 to 2, where the diclofenac-component is present in an amount of from 0.05 up to 0.3 weight-% of the total composition.

6. Use of diclofenac, or a topically acceptable salt thereof, for the manufacture of a topical medicament for the topical treatment of burns including sunburn, which topical medicament is characterized in that diclofenac, or a topically acceptable salt thereof, is the only pharmaceutically active substance therein.

7. Use of diclofenac, or a topically acceptable salt thereof, for the manufacture of a topical medicament for the topical treatment of burns including sunburn, which topical medicament is characterized in that diclofenac, or a topically acceptable salt thereof, is the only pharmaceutically active substance therein, and
which topical medicament is further characterized in that the active substance diclofenac, or a topically acceptable salt thereof, is not in conjunction with a solution of nitrous oxide in a pharmaceutically acceptable earner solvent.

8. Use according to either claim 6 or claim 7, where diclofenac sodium is selected as pharmaceutically active substance.

9. Use according to any one of claims 6 to 8, where the pharmaceutically active substance is present in an amount of from 0.01 up to 2 weight-% of the total composition.

10. Use according to any one of claims 6 to 8, where the diclofenac component is present in an amount of from 0.05 up to 0.3 weight-% of the total composition.

11. Use of diclofenac, or a topically acceptable salt thereof, for the manufacture of a topical medicament for the topical treatment of burns including sunburn, in which medicament diclofenac, or a topically acceptable salt thereof, is present in an amount of from 0.01 up to 0.7 weight-% of the total composition.

12. Use according to claim 11, where diclofenac, diclofenac sodium, diclofenac potassium, diclofenac diethylammonium or diclofenac epolamine is used.

13. Use according to claim 11, where diclofenac sodium, diclofenac potassium, diclofenac diethylammonium or diclofenac epolamine is used.

14. Use according to claim 11, where diclofenac sodium is used.

15. Use of diclofenac, or a topically acceptable salt thereof, for the manufacture of a topical medicament for the topical treatment of burns including sunburn, in which medicament diclofenac, or a topically acceptable salt thereof, is present in an amount of from 0.05 up to 0.3 weight-% of the total composition.

16. Use according to claim 15, where diclofenac, diclofenac sodium, diclofenac potassium, diclofenac diethylammonium or diclofenac epolamine is used.

17. Use according to claim 15, where diclofenac sodium, diclofenac potassium, diclofenac diethylammonium or diclofenac epolamine is used.

18. Use according to claim 15, where diclofenac sodium is used.

19. Use according to any one of claims 1 to 18, where the topical medicament manufactured is in the form of an emulsion-gel, a gel, a transdermal patch or a plaster.

20. Use according to any one of claims 1 to 18, where the topical medicament manufactured is in the form of an emulsion-gel or a transdermal patch.

21. Use according to any one of claims 2, 8, 14 or 18, where the topical medicament manufactured is in the form of an emulsion-gel or a gel.

22. Use according to any one of claims 2, 8, 14 or 18, where the topical medicament manufactured is in the form of an emulsion-gel.
